(19) United States
(12) Patent Application Publication
(10) Pub. No.: US 2026/0284043 A1
(43) Pub. Date: Sep. 24, 2026

(54) COMPOUNDS FOR UPREGULATING UTROPHIN LEVELS IN MUSCLE CELLS AND METHOD OF APPLICATION THEREOF

(71) Applicant: Peptris Technologies Private Limited, Bangalore (IN)

(72) Inventors: Anand Budni, Bangalore (IN); Venkatasubramanian Narayanan, Bangalore (IN); Shridhar Narayanan, Bangalore (IN)

(73) Assignee: Peptris Technologies Private Limited, Bangalore (IN)

(21) Appl. No.: 19/473,523

(22) PCT Filed: Apr. 11, 2024
(86) PCT No.: PCT/IN2024/050379

§ 371 Date: Oct. 8, 2025

(30) Foreign Application Priority Data
Apr. 11, 2023 (IN) 202341026707

(51) Int. Cl. A61K 31/4985; A61K 31/27; A61K 31/40; A61K 31/4196; A61K 31/496; A61K 31/522; A61K 31/573; A61K 31/58; A61K 45/06; A61P 21/00

(52) U.S. Cl. CPC A61K 31/4985; A61K 31/27; A61K 31/40; A61K 31/4196; A61K 31/496; A61K 31/522; A61K 31/573; A61K 31/58; A61K 45/06; A61P 21/00

(57) ABSTRACT

Disclosed herein are compounds for the treatment or management or both of muscular dystrophy. The compounds, according to the embodiments herein, are capable of inhibiting DPP-IV activity, thereby upregulating utrophin levels in muscles, resulting in an overall improvement in muscle function. Embodiments herein also achieve a composition for upregulating utrophin levels in muscle cells.

### CROSS REFERENCE TO RELATED APPLICATION

This application is based on and derives the benefit of Indian Provisional Application 202341026707, the contents of which are incorporated herein by reference.

### TECHNICAL FIELD

Embodiments disclosed herein generally relate to compounds for the treatment or management of muscular dystrophy. More specifically, the invention relates to DPP-IV inhibitors and their use for upregulating utrophin levels in muscle cells for the treatment of muscular dystrophy.

### BACKGROUND

Muscular dystrophy (MD) is a collection of inherited diseases characterized by skeletal muscle weakness and degeneration. Duchenne Muscular dystrophy (DMD) is one of the most common forms of muscular dystrophy, caused by C-chromosome linked recessive mutations in the dystrophin gene. DMD affects approximately 1 in 5000 males globally.

Mutations in the dystrophin gene hinder the production of the muscle isoform of dystrophin, a crucial component of the dystrophin-associated glycoprotein complex (DGC) which serves to bridge the internal cytoskeleton with the surrounding extracellular matrix. Dystrophin plays a pivotal role in providing structural stability to skeletal muscle, preserving strength and flexibility, and safeguarding the sarcolemma from injury induced by muscle contractions. Individuals with dystrophies exhibit either low levels or a complete absence of dystrophin expression, resulting in progressive muscle degeneration, and disruption of the neuromuscular junction organization. The absence of dystrophin also leads to elevated intracellular calcium levels and excessive nitric oxide production, initiating processes such as protein degradation, free radical generation, oxidative stress, inflammation, fibrosis, necrosis, and macrophage activation, ultimately culminating in skeletal muscle dystrophic state, respiratory impairment and cardiomyopathy. Progressive muscle degeneration often leads to loss of ambulation at 8-12 years with premature death at 20-30 years due to respiratory and cardiac complications.

A comprehensive cure for the disease remains elusive despite extensive investigation into the molecular mechanisms of muscular dystrophies, and the currently available treatments primarily offer only supportive care. The management of muscular dystrophy primarily relies on symptomatic treatment that entails physiotherapy and the use of corticosteroids. While corticosteroids can help slow disease progression, they are associated with significant side effects such as weight gain, hyperglycemia, insulin resistance, Cushingoid feature's, short stature, behavioral changes, osteoporosis, and bone fractures.

Therapeutic strategies for muscular dystrophy primarily focus on restoring dystrophin expression using various gene therapy methods such as antisense oligonucleotide-mediated exon skipping, AAV-mediated mini-dystrophin gene delivery, CRISPR/Cas9 genome editing and stop-codon suppression. However, these approaches are mutation-specific and are restricted to only a subset of dystrophy patients. Challenges including concerns about immunological adverse events, toxicities and the necessity for systemic delivery further complicate their use. Hence, it is crucial to identify therapeutic strategies capable of mitigating muscle fiber damage and postponing the onset of disability in muscular dystrophy patients, irrespective of the mutation type.

Upregulation of utrophin, an autosomal homologue sharing structural and functional similarities with dystrophin, offers an alternate therapeutic approach for the treatment of muscular dystrophy. Utrophin is expressed in fetal muscle and various non-skeletal muscle tissues in the adults, including the lungs, kidneys, and liver. Spontaneous compensatory upregulation of utrophin is frequently observed in individuals with muscular dystrophy, as well as in animal models lacking dystrophin. Seminal studies conducted in animal models support utrophin's potential as a functional substitute for dystrophin, suggesting its viability as a therapeutic approach for treating muscular dystrophies. Moreover, therapeutic interventions utilizing small molecules to elevate utrophin levels in the muscles of individuals with muscular dystrophy are unlikely to trigger an immune response or cause adverse side effects.

Utrophin can be upregulated by various signaling pathways, such as AHR-ARNT, TGF-β, HDAC, GLP-1-PGC-1α, GABPα/β and Calcineurin-NFAT-mediated signaling pathways. Proposed strategies for modulating utrophin expression include the utilization of small drugs to enhance its expression at both the transcriptional and translational levels. The long-term implications of therapeutic approaches focusing on utrophin, however, remain uncertain and require further clinical evaluations. For instance, the development program for the small molecule drug Ezutromid, designed to upregulate utrophin, was recently terminated due to its failure to meet endpoints in clinical trials potentially because of self-limiting pharmacokinetic profile of the molecule. Thus, there is currently a lack of evidence for the availability of a therapeutic intervention for clinically upregulating utrophin levels to effectively treat patients with muscular dystrophies.

Therefore, there exists a critical need to identify therapeutic agents with high efficacy, ease of administration, broad applicability, and excellent safety and tolerability profiles for the prevention, treatment, and management of muscular dystrophy.

### Objects

The principal object of the embodiments herein is to provide a compound for the treatment or management or both of muscular dystrophy.

Another object of the embodiments herein is to provide a compound that can upregulate utrophin levels in muscles.

Another object of the embodiments herein is to provide a compound that can activate muscle regeneration and repair.

Another object of the embodiments herein is to provide a compound that can prevent or delay the muscle wasting or muscle degradation.

Another object of the embodiments herein is to provide a compound capable of reducing inflammation, oxidative stress, fibrosis and necrosis in muscles.

Another object of the embodiments disclosed herein is to provide a compound that is readily available, cost-effective, user-friendly, therapeutically effective, sustainable, rapid, and has minimal side effects.

Another object of the embodiments herein is to provide a compound that confers potential protection against neuromuscular diseases.

Another object of the embodiments herein is to provide a compound for the preparation of a medication for the treatment or management or both of muscular dystrophy through above mentioned mechanisms.

Another object of the embodiments herein is to provide a compound with Dipeptidyl Peptidase IV (DPP-IV) inhibitor activity for the treatment or management or both of muscular dystrophy.

Another object of the embodiments herein is to provide a composition for the treatment or management or both of muscular dystrophy.

Another object of the embodiments herein is to provide a method for the treatment or management or both of muscular dystrophy.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, throughout which like reference letters indicate corresponding parts in the various figures. The embodiments herein will be better understood from the following description with reference to the following illustratory drawings. Embodiments herein are illustrated by way of examples in the accompanying drawings, and in which:

FIG. 1 presents the effect of DPP-IV inhibitors on utrophin levels in vitro, according to embodiments as disclosed herein.

FIG. 2 presents a comparison of utrophin upregulation by Sitagliptin and Ezutromid on C2C12 mouse skeletal muscle myoblasts, according to embodiments as disclosed herein.

FIG. 3 is a schematic diagram illustrating the protocol for in vivo studies in D2.mdx mouse model of DMD, according to embodiments as disclosed herein.

FIG. 4 displays the results of the treadmill test in D2.mdx mice wherein FIG. 4A depicts the effect of Sitagliptin in terms of the distance travelled and FIG. 4B depicts the effect of Sitagliptin in terms of time to exhaust, according to embodiments as disclosed herein.

FIGS. 5A and 5B presents the effect of Sitagliptin on normalized grip strength in D2.mdx mice pre-treadmill and post-treadmill, respectively, according to embodiments as disclosed herein.

FIGS. 6A and 6B presents the effect of Sitagliptin on hanging latency in D2.mdx mice in pre-treadmill and post-treadmill, respectively, according to embodiments as disclosed herein.

FIG. 7 presents the effect of Sitagliptin on latency to fall in D2.mdx mice from rotarod test, according to embodiments as disclosed herein.

FIG. 8 presents the effect of Sitagliptin on serum creatinine kinase levels in D2.mdx mice, according to embodiments as disclosed herein.

FIG. 9 presents the 28^{th} day summary of treadmill test, wherein FIG. 9A depicts distance travelled, FIG. 9B depicts time to exhaust,

FIG. 9C depicts normalized grip strength pre-treadmill, FIG. 9D depicts normalized grip strength post-treadmill, FIG. 9E depicts hanging test pre-treadmill,

FIG. 9F depicts hanging test post-treadmill, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

For the purposes of interpreting this specification, the definitions (as defined herein) will apply and whenever appropriate the terms used in singular will also include the plural and vice versa. It is to be understood that the terminology used herein is for the purposes of describing particular embodiments only and is not intended to be limiting. The terms “comprising”, “having” and “including” are to be construed as open-ended terms unless otherwise noted.

The words/phrases “exemplary”, “example”, “illustration”, “in an instance”, “and the like”, “and so on”, “etc.”, “etcetera”, “e.g.,”, “i.e.,” are merely used herein to mean “serving as an example, instance, or illustration.” Any embodiment or implementation of the present subject matter described herein using the words/phrases “exemplary”, “example”, “illustration”, “in an instance”, “and the like”, “and so on”, “etc.”, “etcetera”, “e.g.,”, “i.e.,” is not necessarily to be construed as preferred or advantageous over other embodiments. The terms “comprising”, “having” and “including” are to be construed as open-ended terms unless otherwise noted. The terms “individual”, or “patient” or “subject” or “cell-line” are used herein interchangeably.

It should be noted that elements in the drawings are illustrated for the purposes of this description and ease of understanding of aspects of the embodiments as disclosed herein. The accompanying drawings are used to help easily understand various technical features and it should be understood that the embodiments presented herein are not limited by the accompanying drawings. As such, the present disclosure should be construed to extend to any modifications, equivalents, and substitutes in addition to those which are particularly set out in the accompanying drawings and the corresponding description. Usage of words such as first, second, third etc., or I, II, III, etc., to describe components/elements/steps is for the purposes of this description and should not be construed as sequential ordering/placement/occurrence unless specified otherwise.

Embodiments herein disclose compounds for the preparation of a medicament for the treatment or management, or both of muscular dystrophy. The inventors of this application have shown, for the first time, that inhibiting DPP-IV activity can upregulate utrophin levels in in vitro mouse skeletal muscle cell lines, as well as in vivo in mouse D2-mdx mouse model of Duchenne Muscular Dystrophy (DMD), resulting in an overall improvement in muscle function. The inventors have further illustrated that DPP-IV inhibitors such as Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, and Prusogliptin, typically used for the treatment of type 2 diabetes, can be repurposed to upregulate utrophin levels in muscle cells and can be used for the treatment or management of muscular dystrophy. Accordingly, embodiments herein disclose the use of dipeptidyl peptidase-IV (DPP-IV) inhibitors for the treatment or management or both of muscular dystrophy. Specifically, the inventors have shown that Sitagliptin can upregulate utrophin levels in muscle cells, resulting in an overall improvement in muscle function. Embodiments herein also achieve a composition for upregulating utrophin levels in muscle cells. The composition, according to the embodiments herein, includes at least one DPP-IV inhibitor or its pharmaceutically acceptable salt, solvate or analogue thereof, and optionally at least one pharmaceutically acceptable excipient.

The term “muscular dystrophy” refers to genetically and clinically heterogeneous group of rare neuromuscular diseases caused by mutations in the dystrophin gene, dysferlin gene and associated glycoprotein complex (DAPC/DGC). Muscular dystrophy, as used herein, encompass different categories of muscular dystrophies including, but not limited to, dystroglycanopathy, dysferlinopathy and dystrophinopathy.

Dystroglycanopathy is a collective term referring to muscular dystrophies with abnormal glycosylation of α-dystroglycan (DG), a glycoprotein that interacts with dystrophin or mutations of genes related to Dystroglycan protein complex (DAPC/DGC). Dystroglycanopathy exhibit a broad clinical spectrum, ranging from severe congenital muscular dystrophies, to mild ones, including Fukuyama Congenital muscular dystrophy (FCMD), Myotonic muscular dystrophy, Facioscapulohumeral muscular dystrophy (FSHD1/2), Congenital muscular dystrophy (CMD1C), Limb-girdle muscular dystrophy (LGMD's around 32 variants including LGMDR9/LGMD2I), Emery-Dreiffus muscular dystrophy (EDMD), Muscle-Eye-Brain disease (MEB), Walker-Warburg syndrome (WWS), Calpainopathis or LGMD2A and Oculopharyngeal muscular dystrophy.

Dystrophinopathy covers a spectrum of X-linked muscle disease ranging from mild to severe that includes Duchenne muscular dystrophy, Becker muscular dystrophy, and DMD-associated dilated cardiomyopathy (DCM).

Dysferlinopathy is a disease caused by a dysferlin deficiency due to mutations in the DYSF gene. Dysferlin is a membrane protein in the sarcolemma and is involved in different functions, such as membrane repair and vesicle fusion, T-tubule development and maintenance, Ca²⁺ signaling, and the regulation of various molecules. Dysferlinopathy includes Miyoshi Myopathy type 1 (MMD1) and Limb-Girdle Muscular Dystrophy R2 dysferlin-related (LGMDR2). Accordingly, the compounds of the present invention can be used for the treatment or management or both of muscular dystrophy including, but not limited to, Fukuyama Congenital muscular dystrophy (FCMD), Myotonic muscular dystrophy, Facioscapulohumeral muscular dystrophy (FSHD1/2), Congenital muscular dystrophy (CMD1C), Limb-girdle muscular dystrophy, Emery-Dreiffus muscular dystrophy (EDMD), Muscle-Eye-Brain disease (MEB), Walker-Warburg syndrome (WWS), Calpainopathis or LGMD2A, Oculopharyngeal muscular dystrophy, Duchenne muscular dystrophy, Becker muscular dystrophy, DMD-associated dilated cardiomyopathy (DCM), Miyoshi Myopathy type 1 (MMD1), and Limb-Girdle Muscular Dystrophy R2 dysferlin-related (LGMDR2) muscular dystrophy.

In one embodiment, the muscular dystrophy is Duchenne Muscular Dystrophy (DMD). In another embodiment, the muscular dystrophy is Becker muscular dystrophy (BMD). Both DMD and BMD are characterized by progressive muscle weakness and skeletal degeneration. In DMD patients, dystrophin is virtually absent, whereas BMD patients have 10% to 40% of the normal amount. The increased permeability of the sarcolemma caused by dystrophy often leads to the release of creatine kinase (CK) from muscle fibers. Therefore, an increased level of serum CK is the hallmark of muscle damage. In patients with DMD, CK is markedly elevated compared with the normal range, which has diagnostic value.

Muscular dystrophy, as used herein, also includes atrophy characterized by muscle degeneration or loss of mass often attributed to aging or various diseases such as polio, severe malnutrition, nerve injuries or other neurogenic disorders. Dystrophy typically stems from genetic mutations and entails severe weakness due to insufficient muscle proteins, often with visible muscle weakness and wasting. While atrophy can be mitigated through exercise and lifestyle adjustments, dystrophy, being genetic in nature, is irreversible.

DPP-IV inhibitors, as used herein, refers to molecules that inhibit the activity of dipeptidyl peptidase-IV (DPP-IV) enzyme. DPP-IV is an enzyme expressed on the surface of most cell types and is associated with immune regulation, signal transduction, and apoptosis. DPP-IV enzyme plays a major role in glucose metabolism and is responsible for the degradation of incretins such as glucagon like peptide (GLP-1) and glucose-dependent insulinotropic polypeptide (or Gastric inhibitory polypeptide, GIP). The DPP-IV enzyme possesses five binding sites, namely S1, S2, S1′, S2′, and S2 extensive. Primary interaction with S1 and S2 is crucial for DPP-IV inhibition, with further interactions at S1′, S2′, and the S2 extensive site potentially enhancing inhibition. Examples of DPP-IV inhibitors include, but are not limited to, Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, and Prusogliptin.

DPP-IV inhibitors are categorized based on their interactions with the enzyme into Class 1, Class 2, and Class 3. Class 1 inhibitors, such as vildagliptin and saxagliptin, bind to S1 and S2, representing fundamental inhibitors. Class 2 inhibitors (e.g., Alogliptin and Linagliptin) interact with additional sites (S1′ and S2′), potentially leading to increased inhibition compared to Class 1. Class 3 inhibitors (e.g., Sitagliptin and Teneligliptin) bind to an additional site, S2 extensive, resulting in more extensive DPP-IV inhibition.

The compound for the preparation of a medicament for the treatment or management or both of muscular dystrophy, according to the embodiments herein, includes at least one dipeptidyl peptidase-IV (DPP-IV) inhibitor, their salts, or combinations thereof.

In one embodiment, the compound is Sitagliptin. Sitagliptin or (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine and its phosphate salt are first oral DPP-IV inhibitors approved by the FDA. In one embodiment, the compound is Sitagliptin phosphate monohydrate.

In one embodiment, the compound is Melogliptin. Melogliptin or (2S,4S)-4-fluoro-1-[2-[[(1R,3S)-3-(1,2,4-triazol-1-ylmethyl)cyclopentyl]amino]acetyl]pyrrolidine-2 carbonitrile is a potent, selective and orally bioavailable, cyanopyrrolidine-based DPP-IV inhibitor with hypoglycemic activity.

In one embodiment, the compound is Linagliptin. Linagliptin or 8-[(3R)-3-Aminopiperidin-1-yl]-7-(but-2-yn-1-yl)-3-methyl-1-[(4-methylquinazolin-2-yl)methyl]-3,7-dihydro-1H-purine-2,6-dione is an FDA approved oral antidiabetic drug. Linagliptin differs from other DPP-IV inhibitors in that it has a non-linear pharmacokinetic profile, is not primarily eliminated by the renal system, and obeys concentration dependent protein binding.

In one embodiment, the compound is Vildagliptin. Vildagliptin or (S)-1-[2-(3-Hydroxyadamantan-1-ylamino) acetyl]pyrrolidine-2-carbonitrile is an FDA approved oral antidiabetic agent that enhances pancreatic islet cell responsiveness to glucose.

In one embodiment, the compound is Teneligliptin. Teneligliptin or {(2S,4S)-4-[4-(3-Methyl-1-phenyl-1H-pyrazol-5-yl)-1-piperazinyl]-2-pyrrolidinyl}(1,3-thiazolidin-3-yl)methanone is one of the newer anti-diabetic medications.

It is also within the scope of the invention, to use salts, solvates, derivatives, or analogues of Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, and Prusogliptin.

### Composition

Embodiments herein also achieve a composition for the treatment or management or both of muscular dystrophy. In one embodiment, the composition contains at least one DPP-IV inhibitor or its pharmaceutically acceptable salt, solvate or analogue thereof. DPP-IV inhibitors include, but are not limited to, Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, and Prusogliptin.

In one embodiment, the composition contains a pharmaceutically acceptable salt of DPP-IV inhibitor. Pharmaceutically acceptable salt, as used herein, refers to a salt that retains the biological effectiveness of the free acids and bases of a specified compound and that is not biologically or otherwise undesirable. Pharmaceutically acceptable salt may also refer to a salt that may have an unexpectedly superior biological efficacy or effectiveness when compared to the actual or active pharmaceutical ingredient (API) as well. According to the present invention, pharmaceutically acceptable salts are produced from acidic inorganic or organic compounds, or alkaline inorganic or organic compounds. Salts derived from inorganic bases include aluminum, ammonium, calcium, copper, ferric, ferrous, lithium, magnesium, manganic salts, manganous, potassium, sodium, zinc, and the like. Salts in the solid form may exist in more than one crystal structure and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Salts from inorganic and organic acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one embodiment, the pharmaceutically acceptable salt is a phosphate salt. In one embodiment, the composition contains pharmaceutically acceptable salt of sitagliptin. In one embodiment, the composition contains sitagliptin phosphate monohydrate.

In one embodiment, the composition includes a solvate or analogue of DPP-IV inhibitor. A solvate, as used herein, typically refers to a compound (or a salt thereof), in association with a solvent, such as water. Representative examples include hydrates, hemihydrates, trihydrates and the like. As used herein, the term “analogue” is typically used to denote a compound that has a chemical structure that is substantially similar to the structure of the parent compound, whilst retaining at least some of the biological function of the parent compound. Analogues also include pharmaceutically acceptable salts.

In one embodiment, the composition includes at least one pharmaceutically acceptable excipient. Examples of pharmaceutically acceptable excipients include, but are not limited to mannitol, starch, xylitol, maltodextrin, hydroxypropyl methylcellulose, hydroxypropyl cellulose, ethyl cellulose, microcrystalline cellulose, silicified microcrystalline cellulose, dicalcium phosphate anhydrous, glyceryl behenate, triethyl citrate, polyethylene glycol, croscarmellose sodium, stearic acid, talc, hydrogenated cottonseed oil, magnesium stearate, colloidal silicon dioxide, polysorbate, sodium lauryl sulfate, calcium hydrogen phosphate anhydrous, sodium stearyl fumarate, propyl gallate, poly(vinyl alcohol), macrogol 3350, titanium dioxide, red iron oxide, and yellow iron oxide or mixtures thereof. In one embodiment, the composition may further include a pharmaceutically acceptable carrier, diluent and/or adjuvant. It is also within the scope of the invention that the composition may have additional additives selected from solvents, stabilizers, or suspensions.

The composition may be formulated together or separately with the pharmaceutically acceptable excipients or the carriers. Desirably, a compound of the invention and the pharmaceutically acceptable excipient or the carrier are formulated together for their simultaneous or near simultaneous administration. In one embodiment, the pharmaceutically acceptable excipient or the carrier may be formulated separately with a compound of the invention.

The concentration of the DPP-IV inhibitor in the composition may range from as low as 0.1% of the total amount of the composition up to as high as 100%. In some embodiments the concentration of the DPP-IV inhibitor in the composition is from 1% to 90% by weight. In some embodiments the concentration of the DPP-IV inhibitor in the composition is from 5% to 80% by weight. In some embodiments, the concentration of the DPP-IV inhibitor in the composition is from 10% to 70% by weight. The exact amount will depend upon the any additional materials chosen.

The DPP-IV inhibitor or the composition comprising at least one DPP-IV inhibitor, according to the embodiments herein, can be administered as a monotherapy or in combination with one or more additional therapies. In one embodiment, the DPP-IV inhibitor or the composition including the DPP-IV inhibitor is administered as monotherapy. In one embodiment, the DPP-IV inhibitor or the composition including the DPP-IV inhibitor is administered as combination therapy with one or more additional therapeutic agents. Non-limiting examples of additional therapies that can be used for combination therapy include, but are not limited to, corticosteroid therapy, gene therapy, exon-skipping therapy, immunosuppressant therapy, epigenetic therapy, muscle regeneration therapy and muscle-strengthening therapy.

In one embodiment, combination therapy comprises administering the DPP-IV inhibitor or the composition comprising at least one DPP-IV inhibitor with corticosteroids. Corticosteroid therapy includes the administration of corticosteroids to delay the progression of muscular dystrophy. Examples of corticosteroids that are used in the treatment of dystrophy include, but are not limited to, Prednisone/Prednisolone, Deflazacort (an oxazoline derivative of prednisolone), Vamorolone and combinations thereof. Corticosteroids are administered by two common regimens—daily and intermittent.

In one embodiment, combination therapy comprises administering the DPP-IV inhibitor or the composition comprising at least one DPP-IV inhibitor with exon-skipping therapies. Exon skipping therapy refers to the use of antisense oligonucleotides to slice out selected exons from pre-mRNA, at or next to, the mutation site, to generate a translatable transcript from the mutant of dystrophin gene. The antisense oligonucleotides (AONs) are 20-30 nucleotides in length, designed to target specific pre-mRNA sequences and to skip a specific DMD exon flanking the region of mutation, producing an in-frame but truncated transcript that translate a functional dystrophin protein. Examples of AON agents for exon-skipping therapy include, but are not limited to, Eteplirsen, Golodirsen, Viltolarsen, Casimersen, Drisapersen, tricyclo-DNA (tcDNA), ASO-based therapy and combinations thereof.

In one embodiment, combination therapy comprises administering the DPP-IV inhibitor or the composition comprising at least one DPP-IV inhibitor with epigenetic agents. Epigenetic therapy involves utilizing small molecules or epigenetic modifiers to modify gene activity without changing the gene's coding sequence. Key epigenetic mechanisms, such as DNA methylation or histone modification, play a crucial role in regulating muscle regeneration. Epigenetic therapy includes therapeutic approaches by creating epigenetic drugs designed to target specific chromatin elements within individual signaling pathways. Examples of epigenetic drugs include, but are not limited to, Givinostat, Trichostatin A (TSA), Pan-HDAC inhibitors, HDAC6 inhibitors and combinations thereof.

In one embodiment, combination therapy comprises administering the DPP-IV inhibitor or the composition comprising at least one DPP-IV inhibitor with gene therapy agents. Gene therapy includes, but are not limited to, adeno-associated virus (AAV) vector-mediated gene therapy, with the micro-dystrophin gene being a preferred candidate.

It is also within the scope of the invention to use the DPP-IV inhibitor or the composition comprising at least one DPP-IV inhibitor in combination with muscle re-generation therapies such as AAK1 inhibitors or cAMP enhancing mechanisms, other utrophin up regulators, muscle-strengthening therapies such as aryl hydrocarbon receptor (AhR) antagonists, myostatin inhibitors, muscle Ca²⁺ overload inhibitors like P2X7 antagonist, Store Operated Calcium Entry (SOCE)/Calcium Release Activated Calcium (CRAC) channel inhibitors, anti-inflammatory agents working in the NF-kB pathway signaling targets like NF-κB inhibitors, IKK2/β inhibitors, TBK1 inhibitors, Akt-mTOR pathway inhibitors and anti-fibrotic mechanism pathway agents like TGF-β inhibitors, RIPK1/3 inhibitors, Activin receptor inhibitors, Smad2/3 inhibitors and TAK1 inhibitors and other GLP-1 agonists, and GLP-1 pathway activators.

The combination therapy is administered in a manner and at a dosage effective to increase the production of utrophin and improve muscle function and strength.

The DPP-IV inhibitor or the composition comprising at least one DPP-IV inhibitor of the present invention may be used in combination with one or more other drugs in the treatment, suppression or amelioration of muscular dystrophy, where the combination of the drugs together is safer or more effective than either drug alone. Such other drug(s) may be administered, by a route and in an amount commonly used therefor, contemporaneously, or sequentially with the compounds of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. The combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly.

The DPP-IV inhibitor or the composition comprising at least one DPP-IV inhibitor, according to the embodiments herein, can be formulated for administration by any suitable route, including parenteral (e.g., intravenous, intramuscular), intradermal, cutaneous, subcutaneous, oral, transdermal, transmucosal, topical, nasal, vaginal, intrathecal, epidural, ocular and rectal administration or by injection, or inhalation.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition is preferably sterile and should be fluid to the extent that easy syringability exists. In some embodiments, it will be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, or liquid polyetheylene glycol, and the like), and suitable mixtures thereof. The proper fluidity can be maintained, for example, by the use of a coating such as lecithin, by the maintenance of the required particle size in the case of a dispersion or by the use of surfactants. Prevention of the action of microorganisms can be achieved by incorporation of various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, or sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yield a powder of the active ingredient plus any additional desired ingredient from a previously sterile-filtered solution thereof.

The DPP-IV inhibitor or the composition including the DPP-IV inhibitor may be formulated as tablets, hard or soft capsules, gummy chewables, syrups, elixirs, pills, troches, lozenges, emulsions, dispersible powders or granules, liquids, gels, aqueous or oily suspensions, patches, nano formulations or other suitable forms for oral, parenteral, topical, or inhalation administration. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Suitable tablets may be obtained for example, by mixing at least one of the compounds that may be used in the present invention with known excipients, for example diluents such as microcrystalline cellulose, calcium carbonate, calcium phosphate or lactose, disintegrants such as croscaramellose sodium, HPMC, sodium starch glycolate, binders such as starch or gelatin, guar gum, xanthum gum, lubricants such as magnesium stearate or talc and/or agents. The shapes include round, caplet, flat, oval and bevelled edges with and without embossing.

Capsules like hard or soft gelatin containing the compounds that may be used in the present invention may, for example, be prepared by mixing the active compounds with inert carriers such as lactose or sorbitol and packing them into gelatin capsules. Capsules may be with or without imprinting.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The composition may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soybean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, or sucrose. Such formulations may also contain a demulcent, a preservative and flavoring and coloring agents.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fusidic acid derivatives. Transmucosal administration can be accomplished with nasal sprays or suppositories. The compounds can be prepared in the form of suppositories (e.g., with conventional suppository bases such as cocoa butter and other glycerides) or retention enemas for rectal delivery.

The dosage of the DPP-IV inhibitor or the composition including the DPP-IV inhibitor will vary with the specific compound employed, metabolic stability and length of action of the compound, the route and time of administration, the rate of excretion, the duration of the treatment, severity of the condition, drug combination, the identity of any other therapeutic compounds being administered, the age, body weight, general health, sex, diet, size, and species of the subject, e.g., human patient, and like factors. In general, the dosage of DPP-IV inhibitors in the present composition will be an amount which is the lowest dose effective to produce the desired effect with no or minimal side effects. The effective dose of DPP-IV inhibitors may also be administered as two, three, four, five, six or more sub-doses, administered separately at appropriate intervals throughout the day. An appropriate dosage level will generally be about 10 to 250 mg per day which can be administered in single or multiple doses. Preferably, the dosage level will be about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day. In one embodiment, the composition is administered at a dose between 30 mg/kg per day and 70 mg/kg per day in mice. In one embodiment, the composition is administered at a dose of 50 mg/kg per day in D2.mdx mice. In one embodiment, the broader human dosage of the composition is 10 to 250 mg per day, administered in a single or multiple dosage regimen.

In one embodiment, the DPP-IV inhibitor or the composition including the DPP-IV inhibitor is used for the treatment or management, or both, of muscular dystrophy. Treatment or management includes inhibiting the condition, that is, arresting the development or progression of clinical symptoms, and/or relieving the condition, i.e., causing regression of clinical symptoms. The composition, according to the embodiments herein can be used to manage symptoms of muscular dystrophy such as muscle weakness and wasting and to slow disease progression. The composition can also be used to improve the quality of life in dystrophy patients. In one embodiment, the composition provides a strategy for a dystrophy-specific therapy that in principle is applicable to all patients, i.e., not limited to restricted subsets of patients having mutation-specific muscular dystrophies.

In one embodiment, the DPP-IV inhibitor or the composition including the DPP-IV inhibitor upregulates utrophin expression in muscle cells. Utrophin expression is subject to regulation at multiple steps throughout its synthesis and degradation pathways. Different approaches for modulating utrophin expression include, but are not limited to, direct mechanisms such as gene or protein replacement, and indirect ones, such as transcriptional upregulation of the utrophin promoter, post-transcriptional regulation and protein/mRNA stabilization. Utrophin can be upregulated by various signaling pathways, but not limited to, such as AHR-ARNT, TGF-β, HDAC, GLP-1-PGC-1α, GABPα/β and Calcineurin-NFAT-mediated signaling pathways. In one embodiment, the composition upregulates utrophin expression via inhibiting DPP-IV.

In one embodiment, the DPP-IV inhibitor or the composition including the DPP-IV inhibitor activates PI3K/Akt signaling pathway, which is known to play a role in muscle growth and regeneration. DPP-IV inhibition can activate PI3K/Akt signaling pathway in muscle cells, increasing protein synthesis and muscle fiber size. Another mechanism by which the composition may exert its effects on muscular dystrophies is through the stimulation of mitochondrial biogenesis. In one embodiment, the composition upregulates myogenic factors like myogenin (MyoG) and MyoD. In one embodiment, the composition inhibits inflammation, muscle atrophic factors and thereby reduce muscle wasting. In one embodiment, the composition can reduce muscle fibrosis and necrosis. In an embodiment, the composition has potential therapeutic effects in animal models and clinical trials, indicating its efficacy in humans.

The use of DPP-IV inhibitors as repurposed drug for the upregulation of utrophin has several advantages. DPP-IV inhibitors are potent, well-tolerated, and orally bioavailable drugs with broad applicability, excellent safety and tolerability profiles, making them suitable for long-term use. DPP-IV inhibitors are also proven to be safe for long term use in pediatric (adolescents) and adult population. DPP-IV inhibitors also demonstrate anti-inflammatory and anti-fibrotic properties, along with the ability to prevent muscle wasting and degradation, while also strengthening myofibers.

Embodiments herein also disclose a method for increasing expression of utrophin in a subject in need thereof, the method comprising administering to the subject, a therapeutically effective amount of the DPP-IV inhibitor or the composition comprising at least one DPP-IV inhibitor or its pharmaceutically acceptable salt, and at least one pharmaceutically acceptable excipient.

Embodiments herein also disclose a method for treatment of muscular dystrophy. The method, according to the embodiments herein, include administering to a subject in need of, a therapeutically effective amount of DPP-IV inhibitor or the composition comprising at least one DPP-IV inhibitor or its pharmaceutically acceptable salt, and at least one pharmaceutically acceptable excipient. The term “effective” or “therapeutically effective”, as used herein, refers to amount of a compound that is nontoxic, but is present in a sufficient amount to provide the desired effect at a reasonable benefit/risk ratio for attending any medical treatment. The desired effect can be alleviation of the signs, symptoms, or causes of a disease, or any other desired result in a biological symptom.

The subject is generally a mammal, preferably a human being, male or female, in whom inhibition of dipeptidyl peptidase-IV enzyme activity is desired. In one embodiment, the subject includes a mammal suffering from muscular dystrophy. In one embodiment, muscular dystrophy includes Fukuyama Congenital muscular dystrophy (FCMD), Myotonic muscular dystrophy, Facioscapulohumeral muscular dystrophy (FSHD1/2), Congenital muscular dystrophy (CMD1C), Limb-girdle muscular dystrophy, Emery-Dreiffus muscular dystrophy (EDMD), Muscle-Eye-Brain disease (MEB), Walker-Warburg syndrome (WWS), Calpainopathis or LGMD2A, Oculopharyngeal muscular dystrophy, Duchenne muscular dystrophy, Becker muscular dystrophy, DMD-associated dilated cardiomyopathy (DCM), Miyoshi Myopathy type 1 (MMD1), and Limb-Girdle Muscular Dystrophy R2 dysferlin-related (LGMDR2) muscular dystrophy.

The invention is further described by reference to the following examples by way of illustration only and should not be construed to limit the scope of the embodiments disclosed herein. It will be apparent to those skilled in the art that many modifications, both to materials and methods, may be practiced without departing from the scope of the claimed embodiments.

### Example 1—Utrophin Upregulation by DPP-IV Inhibitors in Vitro

C2C12 myoblast cells are seeded in the well plate with a growth medium (10% FBS and DMEM). After they reached 70% confluence, the cells are added to the differentiation medium (2% HS and DMEM) and differentiated for seven days. Stock solutions are prepared for Sitagliptin and other DPP-IV inhibitors viz., Melogliptin, Linagliptin, Vildagliptin, and Teneligliptin in DMSO. The cells are treated with a final concentration of 10 μM stock solution for 24 hours. RNA isolation is carried out using the Qiagen assay kit and quantified using NanodropRT-PCR for Utrophin Upregulation.

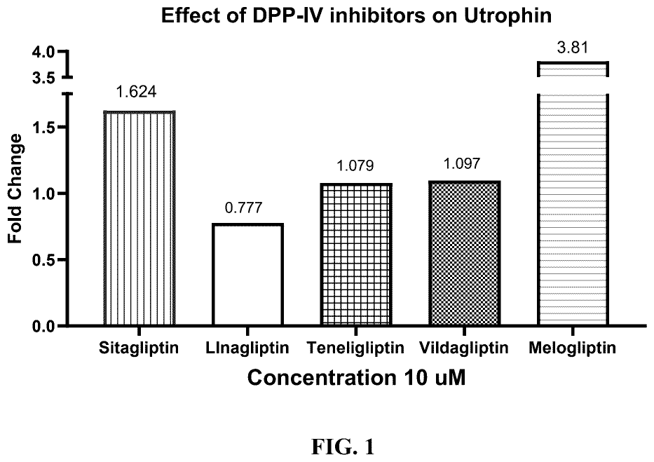

FIG. 1 illustrates the fold change of utrophin at 10 μM concentrations for Sitagliptin, Melogliptin, Linagliptin, Vildagliptin, and Teneligliptin. It is clear that all the DPP-IV inhibitors are capable of upregulating utrophin levels in myoblast cells.

### Example 2—Comparison of Utrophin Upregulation by Sitagliptin and Ezutromid in Vitro

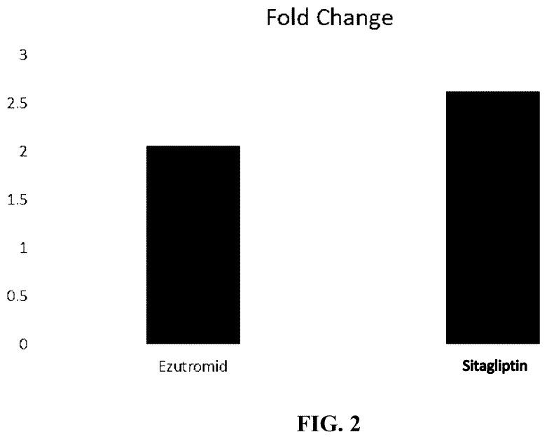

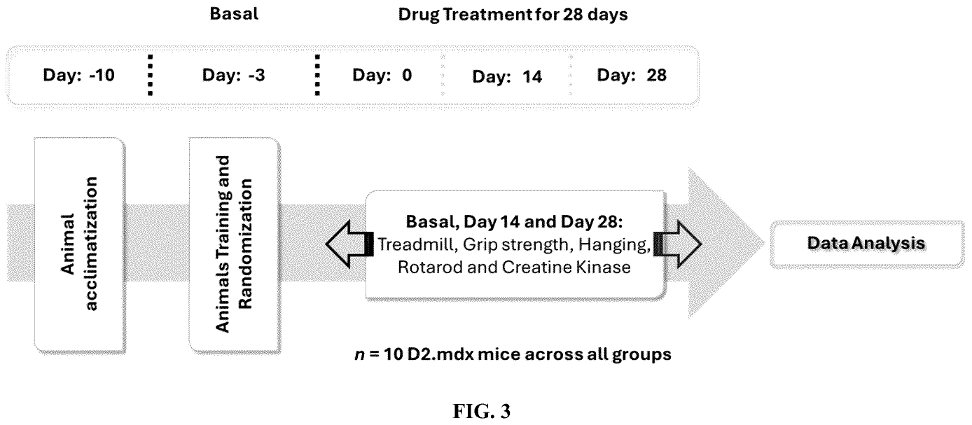

Using the same protocol for Example 1, C2C 12 myoblast cells are treated with sitagliptin for 24 hours. Ezutromid is a known utrophin up-regulator and used as a positive control. RNA isolation and RT-PCR are done to quantify the fold change in utrophin mRNA expression. FIG. 2 presents the fold change in mRNA expression of Utrophin on C2C12 mouse skeletal muscle myoblasts at 10 uM sitagliptin compared to 10 uM Ezutromid. Sitagliptin exhibits superior upregulation of utrophin compared Ezutromid.

### Example 3—in Vivo Studies on D2.mdx Mouse Models

### Drug Administration and Efficacy Evaluation Protocol

All the functional experimental parameters are evaluated in mdx background wild-type mice and in D2.mdx mice (D2.B10-DMD mdx/J mice procured from Jackson laboratories (Strain #013141), Bar Harbor, Maine (ME), USA 04609.

The test item, Sitagliptin 50 mg/kg, is orally (p.o.) administered once daily (q.d.) at a dose volume of 10 mL/kg. Sitagliptin suspended in formulation containing 0.1% Tween 20 and 0.5% Carboxymethyl cellulose (CMC) is administered once daily for 28 days via the oral route. The wild-type control and mdx control groups received the vehicle (0.5% Carboxy methyl cellulose (CMC) containing 0.1% Tween 20) alone. Body weight of the study animals is recorded before the study (pre-dose) and twice weekly/daily throughout the study. Animals are also monitored for clinical signs, mortality, and morbidity.

### Experimental Procedure and Efficacy Evaluation:

D2.mdx and wild-type mice are trained to Treadmill apparatus (Pan Lab, Harvard Instruments, USA) functional test prior to recording their basal (Day 0) performance on the Treadmill apparatus for distance travelled and Time to Exhaust functional parameters. The D2.mdx mice are randomized into MDX-Control and MDX-Sitagliptin groups based on their body weights and their Treadmill functional parameters.

All three group mice (Wild-type, DMD-Control & DMD-Sitagliptin) underwent functional tests at week 0 (Basal—Day 0), week 2 (Day 14) and at the end of the study (week 4 or Day 28). Muscle function is assessed through grip strength test using a Grip strength meter (Orchid Scientific, Model No.: GSM02RS, India), hanging test, treadmill running, and rotarod test (Orchid Scientific, India) in response to Vehicle and Sitagliptin (50 mg/kg, p.o.) treatments. Blood samples are collected for Creatine kinase (CK) analysis 30 min after Treadmill functional test on Day 0, 14 & Day 28. The efficacy of Sitagliptin is evaluated by comparing the functional test parameters and serum CK levels of the treated group with that of the mdx-control group in comparison to the basal value of Wild-type group.

Statistical analyses are performed using GraphPad Prism 10 version software using Two-way ANOVA (multiple comparisons method) followed by Tukey's/Bonferroni ‘t’ test wherein the significance ***p<0.001, **p<0.01 and *p<0.05 vs DMD-Control (Vehicle treated) are applied wherever applicable in the drawings.

FIG. 3 illustrates the protocol adopted for the in vivo studies on D2.mdx mouse models. D2.mdx mouse is a superior DMD model which recapitulates several of the human characteristics of DMD myopathology such as lower hind limb muscle weight, fewer myofibers, increased fibrosis and fat accumulation, and muscle weakness relative to strains with this mutant allele on other genetic backgrounds. D2.mdx mice which are 6-7 weeks old are selected for the 28-day in vivo study. 3 cohorts of 10 animals each are selected-Group-I comprising wild-type mice, Group-II comprising D2.mdx control mice, Group-III comprising D2.mdx mice treated with Sitagliptin (test item). The mice across all groups are subjected to training and randomization for 3 days (Basal, Day −3) followed by treatment for 28 days (Days 0-28). Treadmill, grip strength, hanging, rotarod, and creatine kinase tests are recorded on day 0, day 14 and day 28. The treadmill experiments are conducted on mouse treadmill (Pan Lab, Harvard Instruments, USA). Rotarod test is used as a parameter to record the overall improvement in muscle coordination.

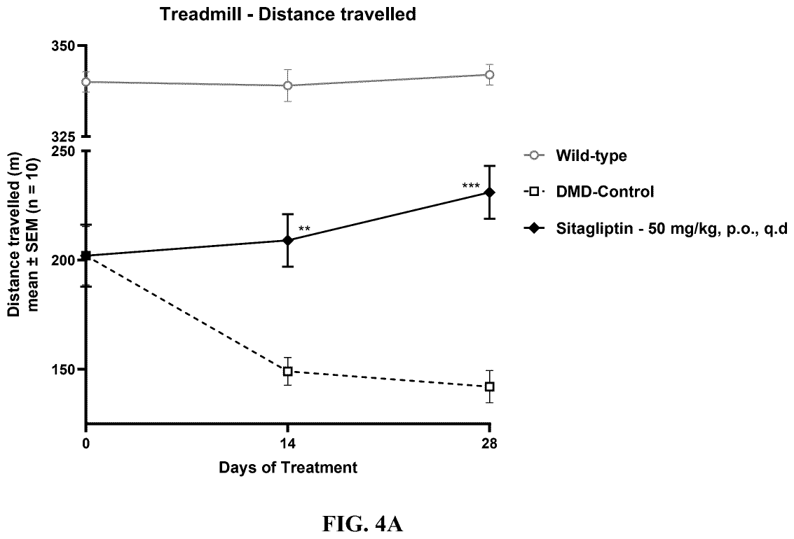

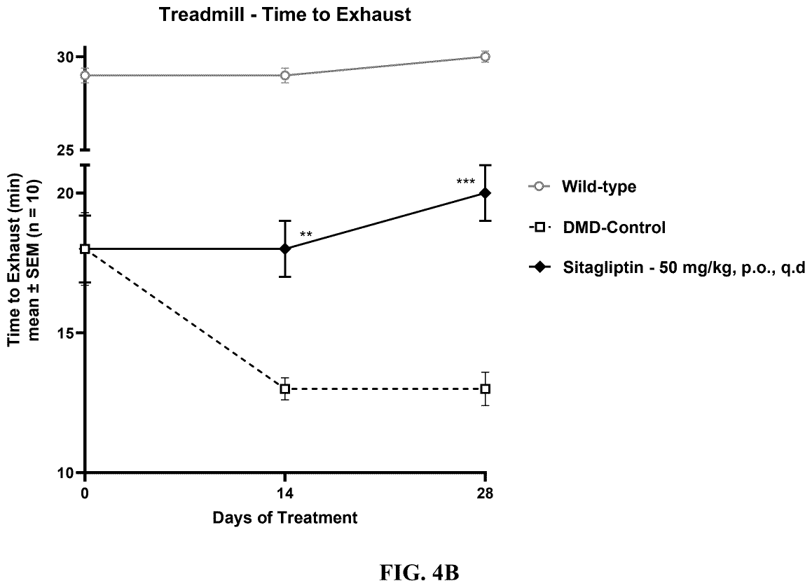

FIG. 4 displays the results of the treadmill test in D2.mdx mice wherein FIG. 4A depicts the effect of Sitagliptin in terms of the distance travelled in 30 mins and FIG. 4B depicts the effect of Sitagliptin in terms of time to exhaust, according to embodiments as disclosed herein. The results suggest that Sitagliptin shows a significant and sustained improvement in distance travelled and time to exhaust over 28 days of treatment.

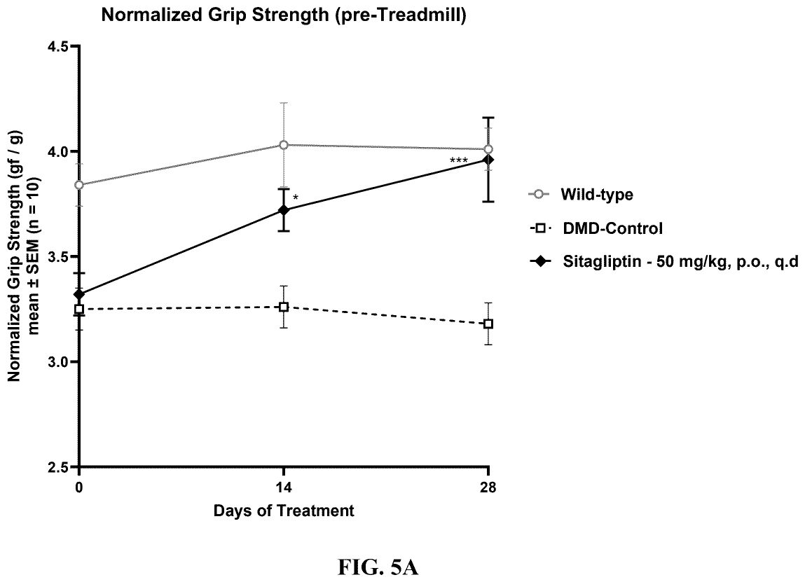

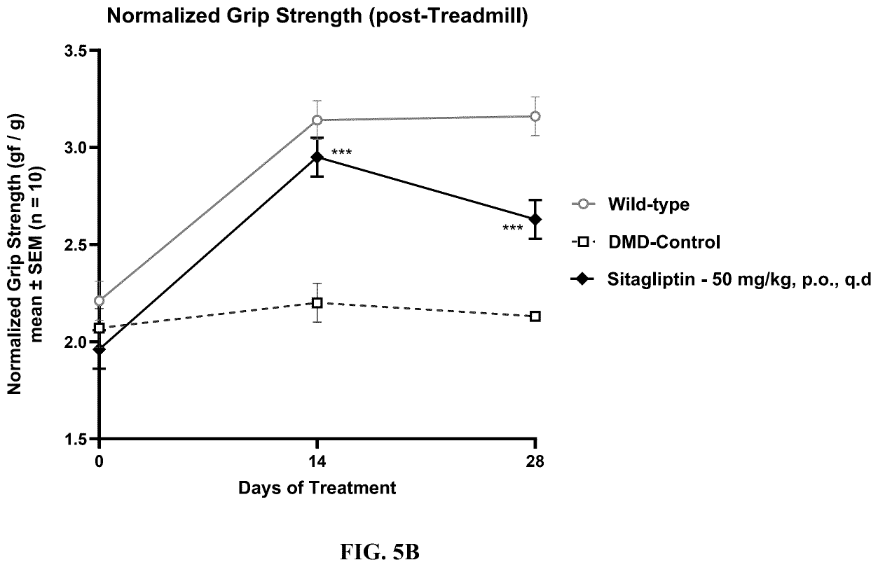

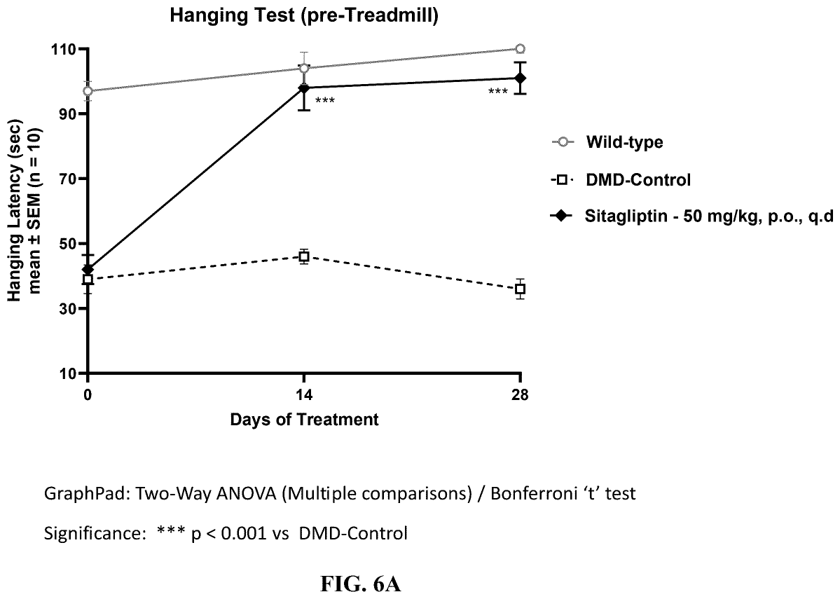

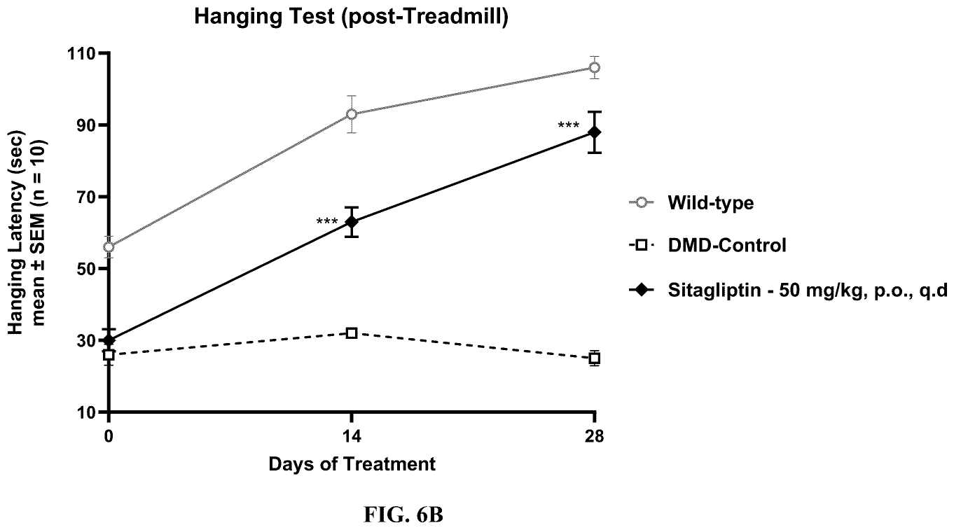

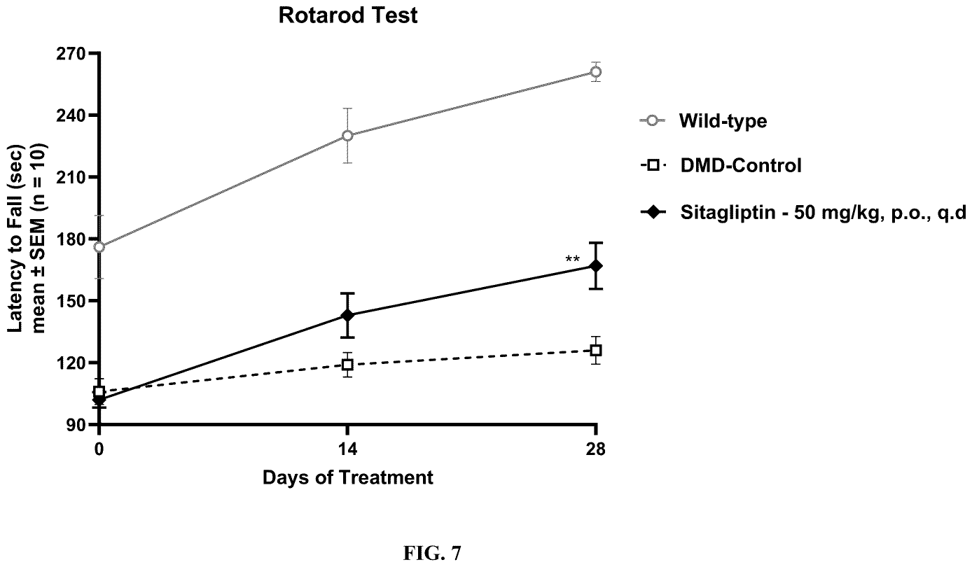

FIGS. 5A and 5B presents the effect of sitagliptin on normalized grip strength in D2.mdx mice pre-treadmill and post-treadmill, respectively, according to embodiments as disclosed herein. Sitagliptin treated mice could grip as well as the wild type mice before being subjected to treadmill stress. Hanging tests also demonstrate that Sitagliptin treated mice perform comparably with wild type mice, irrespective of treadmill stress (FIGS. 6A and 6B). Latency to fall in a Rotarod test indicates that mice treated with Sitagliptin show sustained improvements in overall muscle coordination (FIG. 7).

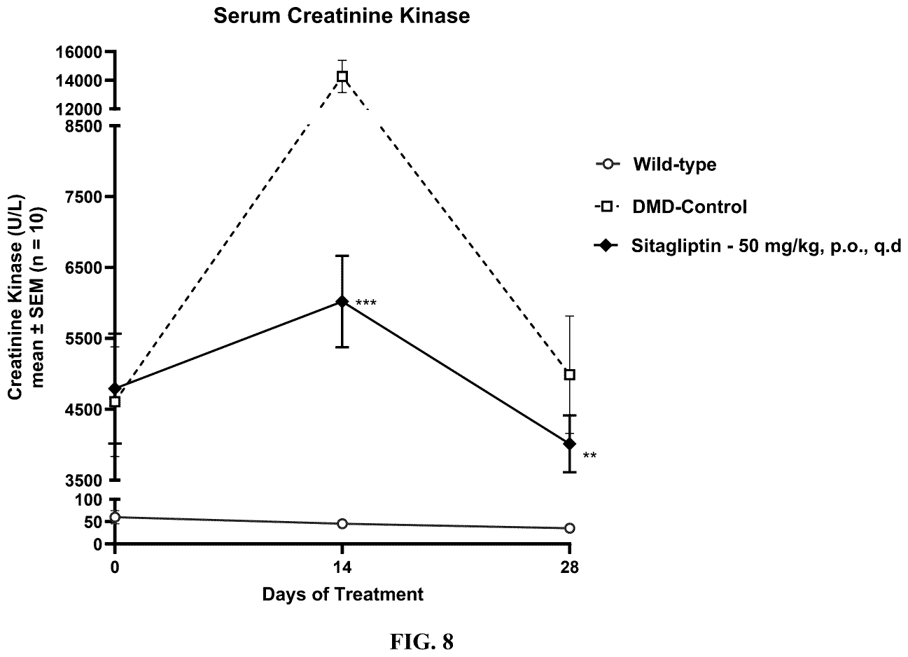

Elevated levels of Serum Creatinine Kinase (CK) in blood sample are an indicative of muscle disintegration caused by muscular dystrophies. Serum Creatinine Kinase level is measured within 30 minutes after the mice are subjected to Treadmill tests. The pattern of Creatinine Kinase changes in mdx control mice is in accordance with published literature. FIG. 8 shows that treatment with Sitagliptin causes significant reductions in Serum Creatinine Kinase levels over 28 days of treatment.

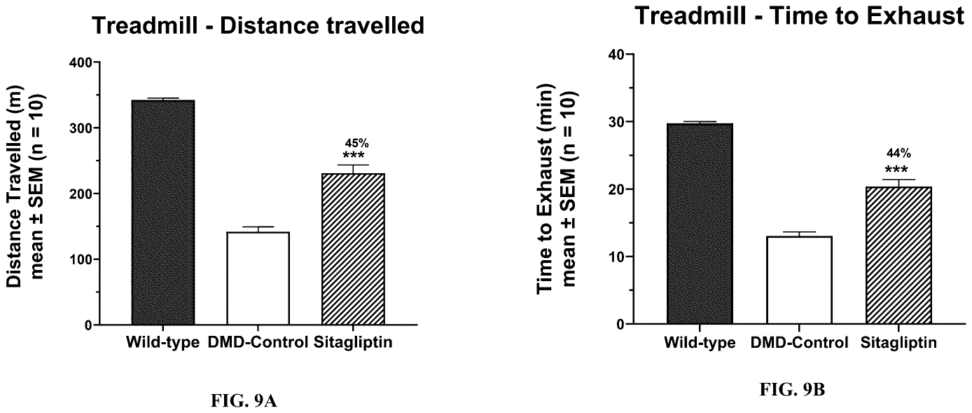

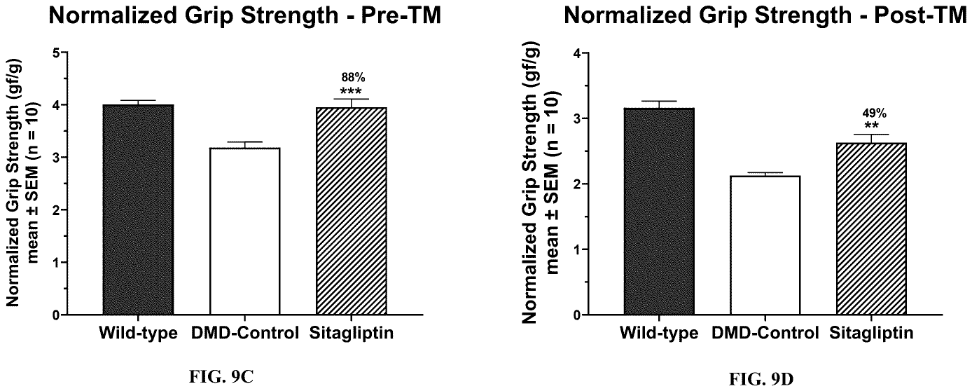

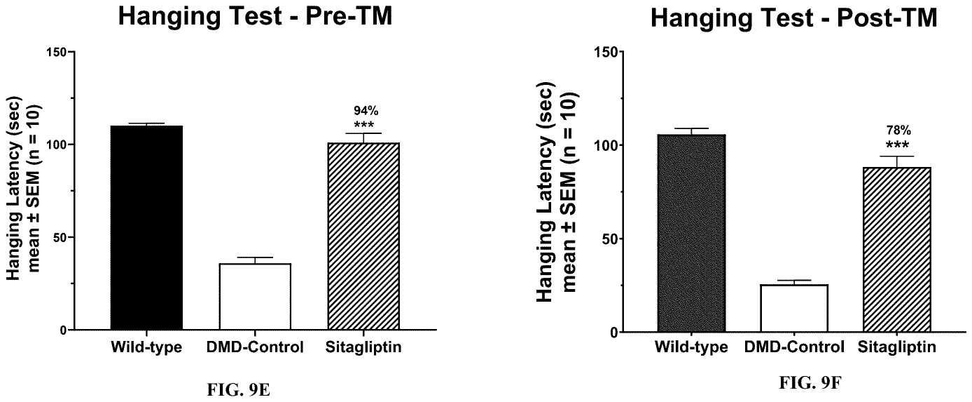

FIG. 9A-9F provides the 28^{th} day summary of the distance travelled, time to exhaust, normalized grip strength pre-and post-treadmill, hanging test pre-and post-treadmill, tests in D2.mdx mouse models. Mice treated with Sitagliptin show a robust improvement in all the functional parameters over the DMD disease control mice. The robust performance is an indicative of an overall improvement in muscle function. These functional results suggest potential utility of Sitagliptin in treatment of DMD.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the scope of the embodiments as described herein.

I/We claim:

1. A compound or its pharmaceutically acceptable salt for the preparation of a medicament for the treatment or management or both of muscular dystrophy, wherein the compound is a DPP-IV inhibitor selected from a group consisting of Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, Prusogliptin and combinations thereof.

2. The compound as claimed in claim 1, wherein the compound is Sitagliptin.

3. The compound as claimed in claim 1, wherein the compound is Sitagliptin phosphate monohydrate.

4. The compound as claimed in claim 1, wherein the compound is Melogliptin.

5. The compound as claimed in claim 1, wherein muscular dystrophy is selected from a group consisting of Duchenne muscular dystrophy, Becker muscular dystrophy, Fukuyama Congenital muscular dystrophy (FCMD), Myotonic muscular dystrophy, Facioscapulohumeral muscular dystrophy (FSHD1/2), Congenital muscular dystrophy (CMD1C), Limb-girdle muscular dystrophy, Emery-Dreiffus muscular dystrophy (EDMD), Muscle-Eye-Brain disease (MEB), Walker-Warburg syndrome (WWS), Calpainopathis or LGMD2A, Oculopharyngeal muscular dystrophy, DMD-associated dilated cardiomyopathy (DCM), Miyoshi Myopathy type 1 (MMD1), and Limb-Girdle Muscular Dystrophy R2 dysferlin-related (LGMDR2) muscular dystrophy.

6. The compound as claimed claim 1, wherein the compound is administered in combination with at least one additional therapy selected from a group consisting of corticosteroid therapy, gene therapy, exon-skipping therapy, immunosuppressant therapy, epigenetic therapy, muscle re-generation therapy and muscle-strengthening therapy.

7. The compound as claimed in claim 6, wherein the corticosteroid therapy comprises administration of at least one corticosteroid selected from a group consisting of Prednisone, Prednisolone, Deflazacort, Vamorolone and combinations thereof.

8. The compound as claimed in claim 6, wherein the exon-skipping therapy comprises administration of at least one agent selected from a group consisting of Eteplirsen, Golodirsen, ASO-based therapy and combinations thereof.

9. The compound as claimed in claim 6, wherein the epigenetic therapy comprises of administration of at least one agent selected from a group consisting of Givinostat, Pan-HDAC inhibitors, HDAC6 inhibitors and combinations thereof.

10. The compound as claimed in claim 1, wherein the dosage of compound is in the range of 10 to 250 mg/day, administered in a single or multiple dosage regimen.

11. The compound as claimed in claim 1, wherein the compound is formulated for intravenous, intramuscular, inhalation, intradermal, cutaneous, subcutaneous, oral, transdermal, transmucosal, topical, nasal, vaginal, intrathecal, epidural, ocular or rectal administration.

12. A composition comprising the compound as claimed in claim 1, or its pharmaceutically acceptable salt, solvates or analogues thereof, and optionally at least one pharmaceutically acceptable excipient.

13. The composition as claimed in claim 12, wherein the compound is a DPP-IV inhibitor selected from a group consisting of Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, Prusogliptin and combinations thereof.

14. The composition as claimed in claim 12, wherein the compound is Sitagliptin.

15. The composition as claimed in claims 12, wherein the compound is Sitagliptin phosphate monohydrate.

16. The composition as claimed in claim 12, wherein the pharmaceutically acceptable excipient is selected from a group consisting of mannitol, starch, xylitol, maltodextrin, hydroxypropyl methylcellulose, hydroxypropyl cellulose, ethyl cellulose, microcrystalline cellulose, silicified microcrystalline cellulose, dicalcium phosphate anhydrous, glyceryl behenate, triethyl citrate, polyethylene glycol, croscarmellose sodium, stearic acid, talc, hydrogenated cottonseed oil, magnesium stearate, colloidal silicon dioxide, polysorbate, sodium lauryl sulfate, calcium hydrogen phosphate anhydrous, sodium stearyl fumarate, propyl gallate, poly(vinyl alcohol), macrogol 3350, titanium dioxide, red iron oxide, and yellow iron oxide and mixtures thereof.

17. The composition as claimed in claim 12, wherein the composition is administered in combination with at least one additional therapy selected from a group consisting of corticosteroid therapy, gene therapy, exon-skipping therapy, immunosuppressant therapy, epigenetic therapy, muscle re-generation therapy and muscle-strengthening therapy.

18. The composition as claimed in claim 17, wherein the corticosteroid therapy comprises administration of at least one corticosteroid selected from a group consisting of Prednisone, Prednisolone, Deflazacort, Vamorolone and combinations thereof.

19. The composition as claimed in claim 17, wherein the exon-skipping therapy comprises administration of at least one agent selected from a group consisting of Eteplirsen, Golodirsen, ASO-based therapy and combinations thereof.

20. The composition as claimed in claim 17, wherein the epigenetic therapy comprises of administration of at least one agent selected from a group consisting of Givinostat, Pan-HDAC inhibitors, HDAC6 inhibitors and combinations thereof.

21. The composition as claimed in claim 12, wherein the dosage of the composition is in the range of 10 to 250 mg/day, administered in a single or multiple dosage regimen.

22. The composition as claimed claim 12, wherein the composition is formulated for intravenous, intramuscular, inhalation, intradermal, cutaneous, subcutaneous, oral, transdermal, transmucosal, topical, nasal, vaginal, intrathecal, epidural, ocular or rectal administration.

23. A method for treatment of muscular dystrophy including administering to a subject in need of, a therapeutically effective amount of the compound as claimed claim 1.

24. The method as claimed in claim 23, wherein the compound is administered in combination with at least one additional therapy selected from a group consisting of corticosteroid therapy, gene therapy, exon-skipping therapy, immunosuppressant therapy, epigenetic therapy, muscle re-generation therapy and muscle-strengthening therapy.

25. The method as claimed in claim 24, wherein the corticosteroid therapy comprises administration of at least one corticosteroid selected from a group consisting of Prednisone, Prednisolone, Deflazacort, Vamorolone and combinations thereof.

26. The method as claimed in claim 24, wherein the exon-skipping therapy comprises administration of at least one agent selected from a group consisting of Eteplirsen, Golodirsen, ASO-based therapy and combinations thereof.

27. The method as claimed in claim 24, wherein the epigenetic therapy comprises of administration of at least one agent selected from a group consisting of Givinostat, Pan-HDAC inhibitors, HDAC6 inhibitors and combinations thereof.

28. The method as claimed in claims 23, wherein muscular dystrophy is selected from a group consisting of Duchenne muscular dystrophy, Becker muscular dystrophy, Fukuyama Congenital muscular dystrophy (FCMD), Myotonic muscular dystrophy, Facioscapulohumeral muscular dystrophy (FSHD1/2), Congenital muscular dystrophy (CMD1C), Limb-girdle muscular dystrophy, Emery-Dreiffus muscular dystrophy (EDMD), Muscle-Eye-Brain disease (MEB), Walker-Warburg syndrome (WWS), Calpainopathis or LGMD2A, Oculopharyngeal muscular dystrophy, DMD-associated dilated cardiomyopathy (DCM), Miyoshi Myopathy type 1 (MMD1), and Limb-Girdle Muscular Dystrophy R2 dysferlin-related (LGMDR2) muscular dystrophy.

29. A method for upregulating utrophin level in muscle cells including administering to a subject in need of, a therapeutically effective amount of the compound as claimed in claim 1.

30. The method as claimed in claim 29, wherein the compound is administered in combination with at least one additional therapy selected from a group consisting of corticosteroid therapy, gene therapy, exon-skipping therapy, immunosuppressant therapy, epigenetic therapy, muscle re-generation therapy and muscle-strengthening therapy.

31. The method as claimed in claim 30, wherein the corticosteroid therapy comprises administration of at least one corticosteroid selected from a group consisting of Prednisone, Prednisolone, Deflazacort, Vamorolone and combinations thereof.

32. The method as claimed in claim 30, wherein the exon-skipping therapy comprises administration of at least one agent selected from a group consisting of Eteplirsen, Golodirsen, ASO-based therapy and combinations thereof.

33. The method as claimed in claim 30, wherein the epigenetic therapy comprises of administration of at least one agent selected from a group consisting of Givinostat, Pan-HDAC inhibitors, HDAC6 inhibitors and combinations thereof.

34. Use of the compound as claimed in claim 1, for the treatment of muscular dystrophy.

35. The use as claimed in claim 34, wherein the compound is a DPP-IV inhibitor selected from a group consisting of Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, Prusogliptin and combinations thereof.

36. The use as claimed in claim 34, wherein the compound is Sitagliptin.

37. The use as claimed in claim 34, wherein the compound is Sitagliptin phosphate monohydrate.

38. The use as claimed in claim 34, wherein muscular dystrophy is selected from a group consisting of Duchenne muscular dystrophy, Becker muscular dystrophy, Fukuyama Congenital muscular dystrophy (FCMD), Myotonic muscular dystrophy, Facioscapulohumeral muscular dystrophy (FSHD1/2), Congenital muscular dystrophy (CMD1C), Limb-girdle muscular dystrophy, Emery-Dreiffus muscular dystrophy (EDMD), Muscle-Eye-Brain disease (MEB), Walker-Warburg syndrome (WWS), Calpainopathis or LGMD2A, Oculopharyngeal muscular dystrophy, DMD-associated dilated cardiomyopathy (DCM), Miyoshi Myopathy type 1 (MMD1), and Limb-Girdle Muscular Dystrophy R2 dysferlin-related (LGMDR2) muscular dystrophy.

39. The use as claimed in claim 34, wherein the compound is administered in combination with at least one additional therapy selected from a group consisting of corticosteroid therapy, gene therapy, exon-skipping therapy, immunosuppressant therapy, epigenetic therapy, muscle re-generation therapy and muscle-strengthening therapy.

40. The use as claimed in claim 39, wherein the corticosteroid therapy comprises administration of at least one corticosteroid selected from a group consisting of Prednisone, Prednisolone, Deflazacort, Vamorolone and combinations thereof.

41. The use as claimed in claim 39, wherein the exon-skipping therapy comprises administration of at least one agent selected from a group consisting of Eteplirsen, Golodirsen, ASO-based therapy and combinations thereof.

42. The use as claimed in claim 39, wherein the epigenetic therapy comprises of administration of at least one agent selected from a group consisting of Givinostat, Pan-HDAC inhibitors, HDAC6 inhibitors and combinations thereof.

43. The use as claimed in claim 34, wherein the dosage of compound is in the range of 10 to 250 mg/day, administered in a single or multiple dosage regimen.

44. The use as claimed in claim 34, wherein the compound is formulated for intravenous, intramuscular, inhalation, intradermal, subcutaneous, oral, transdermal, transmucosal, or rectal administration.

45. Use of the compound as claimed in claim 1, for upregulating utrophin levels in muscle cells.

46. The use as claimed in claim 45, wherein the compound is a DPP-IV inhibitor selected from a group consisting of Sitagliptin, Melogliptin, Linagliptin, Vildagliptin, Teneligliptin, Saxagliptin, Alogliptin, Anagliptin, Gemigliptin, Trelagliptin, Omarigliptin, Evogliptin, Gosogliptin, Retagliptin, Cofrogliptin, Fotagliptin, Prusogliptin and combinations thereof.

47. The use as claimed in claim 45, wherein the compound is Sitagliptin.

48. The use as claimed in claim 45, wherein the compound is Sitagliptin phosphate monohydrate.

49. The use as claimed in claim 45, wherein the compound is administered in combination with at least one additional therapy selected from a group consisting of corticosteroid therapy, gene therapy, exon-skipping therapy, immunosuppressant therapy, epigenetic therapy, muscle re-generation therapy and muscle-strengthening therapy.

50. The use as claimed in claim 49, wherein the corticosteroid therapy comprises administration of at least one corticosteroid selected from a group consisting of Prednisone, Prednisolone, Deflazacort, Vamorolone and combinations thereof.

51. The use as claimed in claim 49, wherein the exon-skipping therapy comprises administration of at least one agent selected from a group consisting of Eteplirsen, Golodirsen, ASO-based therapy and combinations thereof.

52. The use as claimed in claim 49, wherein the epigenetic therapy comprises of administration of at least one agent selected from a group consisting of Givinostat, Pan-HDAC inhibitors, HDAC6 inhibitors and combinations thereof.

53. The use as claimed in claim 45, wherein the dosage of compound is in the range of 10 to 250 mg/day, administered in a single or multiple dosage regimen.

54. The use as claimed in claim 45, wherein the compound is formulated for intravenous, intramuscular, inhalation, intradermal, subcutaneous, oral, transdermal, transmucosal, or rectal administration.
